(19) United States
(12) Patent Application Publication
(10) Pub. No.: US 2016/0000725 A1
(43) Pub. Date: Jan. 7, 2016

(54) Nanoparticle- and Drug-Containing Polymersomes for Medical Applications

(71) Applicant: Northeastern University, Boston, MA (US)

(72) Inventors: Benjamin Geilich, Brookline, MA (US); Thomas Webster, Barrington, RI (US)

(21) Appl. No.: 14/656,261

(22) Filed: Mar. 12, 2015

Related U.S. Application Data
(60) Provisional application No. 61/951,638, filed on Mar. 12, 2014.

(51) Int. Cl. A61K 9/51; A61K 9/00; A61K 31/43

(52) U.S. Cl. CPC A61K 9/5138; A61K 31/43; A61K 9/0019

(57) ABSTRACT

Provided are polymersomes for co-delivery of hydrophobic metallic nanoparticles and pharmaceutical agents and suspensions of such polymersomes. Also provided are methods of making such polymersomes and suspensions of polymersome and methods of using the same to treat diseases or conditions.

### CROSS REFERENCE TO RELATED APPLICATIONS

This application claims the benefit of U.S. Provisional Application No. 61/951,638, filed Mar. 12, 2014 and entitled “Synthesis and Design of a Nanosome Particle Platform for Medical Applications,” which is hereby incorporated by reference in its entirety.

### STATEMENT REGARDING FEDERALLY SPONSORED RESEARCH OR DEVELOPMENT

This invention was developed with financial support from Grant No. DGE-096843 from National Science Foundation. The U.S Government has certain rights in the invention.

### BACKGROUND

Antibiotics have been extensively used since their commercialization in the 1930s to treat patients suffering from a wide variety of infectious diseases. However, antibiotics have been used so prevalently over the last 80 years that the bacteria they were designed to kill have begun to evolve and adapt, rendering these drugs ineffective.^{1,2} According to the Center for Disease Control's 2013 report on antibiotic resistance in the United States, at least 2 million people acquire serious infections from antibiotic resistant bacteria each year, and over 23,000 die as a direct result.³ Even when alternative treatments exist, patients with antibiotic-resistant infections have significantly higher mortality rates, and survivors often have increased hospital stays and long-term complications. These infections cost an estimated $20 billion in excess direct healthcare expenses.³ Infections caused by Gram-negative bacteria are particularly difficult to treat because their robust and hydrophobic outer lipopolysaccharide membrane helps to impede the influx of drugs into the cell.⁴ Of additional concern is the appearance of bacterial strains that are resistant to multiple types of antibiotics (known as multi-drug resistant or MDR strains). Clinicians are now discovering examples of bacteria with such diverse antibiotic resistance that no available drug can successfully treat the infections they cause.⁴ In essence, the near future will bring a new generation of “super bugs” that scientists and doctors do not know how to kill effectively. Unfortunately, the number of new antibiotic drugs in the pipeline has also been rapidly decreasing, largely due to the fact that new drugs are extremely expensive to bring to market, and antibiotics are less financially lucrative to develop when compared to treatments for chronic conditions.⁵ Thus, the need to develop alternative strategies to treat such antibiotic-resistant bacteria, while still utilizing existing drugs, has never been more urgent than today.

Over the past decade, interest in using nanomedicine-based approaches to combat difficult infections has rapidly grown due to the many advantages offered over conventional treatment with free antibiotics. This study explored encapsulating the drug inside nano-sized structures called polymersomes (that is, artificial vesicles made from biodegradable, high molecular weight, amphiphilic block co-polymers). These vesicles typically display a spherical morphology and are composed of hydrated hydrophilic coronas both at the inside and outside of a hydrophobic polymer membrane.⁶ This allows for hydrophilic bioactive materials to be loaded into the particle's aqueous core, and hydrophobic bioactive materials to be loaded into the particle's membrane bilayer. Just loading these compounds into carriers can provide many benefits over treatment with free drugs. For example, encapsulation of antibiotics has been shown to protect the drug from critical bacterial resistance mechanisms such as degradation by β-lactamase enzymes.⁷ In one such study, Nacucchio et al. found that the liposomal encapsulation of piperacillin prevented staphylococcal β-lactamases from hydrolyzing the drug.⁸ Additionally, encapsulation has been demonstrated to facilitate a longer and sustained contact time between the antibiotic and the bacterial cell membrane.⁹

Metallic nanoparticles have long been investigated as potential antibacterial agents due to their many unique physiochemical properties which are not present at the macro scale.¹⁰ Among these metals, silver is perhaps the best known for its antimicrobial effects. Hippocrates noted its ability to enhance wound healing and preserve food and water as early as 400 BC, and many products taking advantage of these properties are available commercially today.^{11,12} In addition, recent studies have shown that there may even be a synergistic effect when silver nanoparticles and antibiotics are used simultaneously to treat a Gram-negative infection.¹³⁻¹⁵ However, it is unknown whether a combined treatment is sufficient to overcome bacteria which display genetic antibiotic resistance. Researchers have also theorized that nanoparticles with hydrophobic functionalization can intercalate into lipid membranes and cause disruption, whereas their hydrophilic counterparts can only adsorb to the cell surface.¹⁶ The difficulty of successfully delivering hydrophobic nanoparticles without significant aggregation in an aqueous environment has limited the investigation of such nanoparticles for antibacterial applications.

### SUMMARY OF THE INVENTION

In one aspect, the invention includes a polymersome including: a membrane having (1) a hydrophobic interior and hydrophilic inner and outer surfaces, the membrane containing an amphiphilic block copolymer comprising a hydrophobic block and a hydrophilic block, wherein the interior of the membrane comprises the hydrophobic block and the inner and outer surfaces of the membrane comprise the hydrophilic block, and (2) one or more hydrophobic metallic nanoparticles disposed in the interior of the membrane; and an aqueous lumen comprising a pharmaceutical agent.

In some embodiments, the hydrophobic metallic nanoparticles include aluminum, calcium, cerium, copper, gold, iron, lithium, magnesium, manganese, platinum, selenium, silver, titanium, tungsten, vanadium, or zinc. In some embodiments, the hydrophobic metallic nanoparticles include silver. In some embodiments, the hydrophobic metallic nanoparticles have an average diameter of from about 2 nm to about 10 nm. In some embodiments, the hydrophobic metallic nanoparticles are functionalized with an alkanethiol.

In some embodiments, the amphiphilic block copolymer is a diblock copolymer. In some embodiments, the diblock copolymer includes polyethylene glycol. In some embodiments, the diblock copolymer includes poly(lactic acid).

In some embodiments, the pharmaceutical agent is an antibiotic. In some embodiments, wherein the pharmaceutical agent is ampicillin.

In some embodiments, the polymersome has a diameter from about 95 nm to about 115 nm. In some embodiments, the polymersome has from about 1 to about 20 nanoparticles.

In some embodiments, the hydrophobic metallic nanoparticles contain silver, the pharmaceutical agent is ampicillin, and the mass ratio of silver:ampicillin in the polymersome is from about 1:1 to about 5:1.

In another aspect, the invention includes a method of making polymersomes, the method including the steps of: providing a suspension of hydrophobic metallic nanoparticles and an amphiphilic block copolymer in an organic solvent; and passing the suspension through an atomizer into an aqueous solution comprising a pharmaceutical agent.

In some embodiments, at least 90% of the polymersomes made by the method have a diameter from about 95 nm to about 115 nm. In some embodiments, at least 90% the polymersomes made by the method comprise from about 1 to about 20 nanoparticles. In some embodiments, the hydrophobic metallic nanoparticles include silver, the pharmaceutical agent is ampicillin, and the mass ratio of silver:ampicillin in the nanoparticles is from about 1:1 to about 5:1.

In another aspect, the invention includes a method of treating a disease or condition, the method including administering to a subject in thereof a polymersome, the polymersome including: a membrane having a hydrophobic interior and hydrophilic inner and outer surfaces, the membrane including (i) an amphiphilic block copolymer including a hydrophobic block and a hydrophilic block, wherein the interior of the membrane includes the hydrophobic block and the inner and outer surfaces of the membrane include the hydrophilic block, and (ii) one or more hydrophobic metallic nanoparticles in the interior of the membrane; and an aqueous lumen comprising a pharmaceutical agent.

In some embodiments, the polymersome is administered by a parenteral route. In some embodiment, the parenteral route is intravascular administration, peri- and intra-tissue administration, subcutaneous injection or deposition, subcutaneous infusion, intraocular administration, or direct application at or near the site of neovascularization. In some embodiments, the polymersome is administered topically, orally, or intranasally.

In some embodiments, the disease or condition is a bacterial infection, viral infection, cancer, mental disorders such as manic depression, and inflammatory diseases such as rheumatoid arthritis.

In another aspect, the invention includes a kit for treating or preventing a microbial infection, the kit including: an aqueous suspension of polymersomes including (i) a membrane having a hydrophobic interior and hydrophilic inner and outer surfaces, the membrane including (A) an amphiphilic block copolymer comprising a hydrophobic block and a hydrophilic block, wherein the interior of the membrane comprises the hydrophobic block and the inner and outer surfaces of the membrane comprise the hydrophilic block and (B) one or more hydrophobic metallic nanoparticles in the interior of the membrane, and (ii) an aqueous lumen comprising a pharmaceutical agent; and instructions for use.

In another aspect, the invention includes a method of imaging a population of cells or molecules in a subject, the method including administering to a subject in thereof an aqueous suspension of polymersomes, the polymersomes including: a membrane having a hydrophobic interior and hydrophilic inner and outer surfaces, the membrane including (i) an amphiphilic block copolymer including a hydrophobic block and a hydrophilic block, wherein the interior of the membrane includes the hydrophobic block and the inner and outer surfaces of the membrane include the hydrophilic block, and (ii) one or more hydrophobic metallic nanoparticles in the interior of the membrane; and an aqueous lumen containing a pharmaceutical agent and/or an imaging agent, wherein the hydrophobic metallic nanoparticles and/or the imaging agent is capable of being detected and forming an image of said population of cells or molecules in said subject.

### BRIEF DESCRIPTION OF THE DRAWINGS

FIG. 1 is a schematic of a polymersome with a membrane containing block copolymers and metallic nanoparticles and an aqueous lumen containing a pharmaceutical agent.

FIG. 2 is a schematic of an embodiment of the method of making a polymersome of the invention. An organic solution containing mPEG-PDLLA copolymers and hydrophobic silver nanoparticles is passed through an atomizer into an aqueous solution containing an antibiotic with constant stirring of the aqueous solution. The organic solvent and unencapsulated antibiotic is then removed from the aqueous solution by dialysis, leaving an aqueous suspension of polymersomes that have silver nanoparticles embedded in their membranes and the antibiotic in their lumens.

FIG. 3A shows transmission electron micrographs of silver nanoparticle-containing polymersomes prepared using an atomizer according to a method of the invention. Inset is a higher magnification view. Scale bars=100 nm. FIG. 3B shows a transmission electron micrograph of silver nanoparticle-containing polymersomes prepared using a needle. Scale bar=100 nm.

FIG. 4 is an image of vials containing suspensions of silver nanoparticle-containing polymersomes prepared using either an atomizer according to a method of the invention (left vial) or a needle (right vial).

FIG. 5A shows transmission electron micrographs of polymersomes with 5 nm silver nanoparticles (dark dots) embedded within their membranes. Inset is a higher magnification view. Scale bars=100 nm. FIG. 5B is a graph showing the size distribution of the polymersomes containing silver nanoparticles. Polymersome size was measured using dynamic light scattering at 25° C. Results indicate an average particle size of 104.3 nm±15.6 nm. FIG. 5C is a graph showing the distribution of polymersomes with various numbers of silver nanoparticles per polymersome. Polymersomes were analyzed in TEM images. Results indicate an average of 9.29±6.07 silver nanoparticles were loaded per polymersome. FIG. 5D is a graph of final (post-dialysis) concentration of ampicillin in a suspension of silver nanoparticle-containing polymersomes vs. the initial (predialysis) concentration of ampicillin in the suspension. Polymersomes were formed in the presence of 300 μg mL⁻¹, 500 μg mL⁻¹, and 800 μ/g mL⁻¹ ampicillin, and final concentrations of ampicillin in polymersome suspensions were 70 μ/g mL⁻¹±7.0 μg mL⁻¹, 110 μg mL⁻¹±7.2 μg mL⁻¹, and 110 μg mL⁻¹±9.0 μg mL⁻¹, respectively. Values represent the mean±standard deviation, N=3. The respective loading efficiencies were 23%, 22%, and 20%. The final silver:ampicillin mass ratios of the polymersomes in these suspensions were 1:0.28, 1:0.44, and 1:0.64, respectively.

FIG. 6 shows graphs of growth of bacterial cells on plates after treatment with various concentrations of ampicillin-loaded, silver nanoparticle-containing polymersomes. 100 μL of cell suspension containing 10⁶, 10⁵, 10⁴, or 10³ CFU/ml, as indicated, was mixed with 100 μL, 75 μL, 50 μL, or 25 μL of ampicillin-loaded, silver nanoparticle-containing polymersomes, as indicated, in a final volume of 200 μL and incubated for 24 hours at 37° C. 25 μL of this mixture was plated on tryptic soy agar medium and incubated overnight at 37° C. “1” indicates plates that formed a continuous film, “0.5” indicates plates that formed individual colonies, and “0” indicates plates that had no growth. Polymersomes in upper graph were made in the presence of 200 μg/mL ampicillin, polymersomes in middle graph were made in the presence of 500 μg/mL ampicillin, and polymersomes in lower graph were made in the presence of 1 mg/mL ampicillin. Bacterial growth values were averaged from three trials (N=3).

FIG. 7A is a graph of OD₆₀₀ vs. time for bacterial cells cultured in the presence of various concentrations ampicillin-loaded, silver nanoparticle-containing polymersomes having a silver:ampicillin mass ratio of 1:0.28. Cultures were inoculated with ampicillin-resistant E. coli at 10⁶ CFU mL⁻¹. The indicated concentrations of ampicillin in the polymersome suspension are used as a proxy for polymersome concentrations because all ampicillin was contained inside the polymersomes. Values represent the mean±standard deviation, N=3. FIG. 7B is a graph of OD₆₀₀ vs. time for bacterial cells cultured in the presence of various concentrations ampicillin-loaded, silver nanoparticle-containing polymersomes having a silver:ampicillin mass ratio of 1:0.44. Other details are the same as in FIG. 7A. FIG. 7C is a graph of OD₆₀₀ vs. time for bacterial cells cultured in the presence of various concentrations ampicillin-loaded, silver nanoparticle-containing polymersomes having a silver:ampicillin mass ratio of 1:0.64. Other details are the same as in FIG. 7A. FIG. 7D is a graph of the time required for bacterial cultures to enter exponential growth phase vs. silver concentration as delivered in polymersomes having silver:ampicillin mass ratios of 1:0.28, 1:0.44, and 1:0.64. Data from FIGS. 6A-6C were used for the graph. FIG. 7E is a graph of ampicillin concentration vs. degree of synergy during the log-linear growth phase as determined at 13.3 hours by the Bliss Independence model. FIG. 7F is a graph of silver concentration vs. degree of synergy during the log-linear growth phase as determined at 13.3 hours by the Bliss Independence model. FIG. 7G is graph of OD₆₀₀ vs. time for bacterial cells cultured in the presence of PBS alone, free ampicillin at 200 μg mL⁻¹, unloaded silver-free polymersomes plus free ampicillin at 200 μg mL⁻¹, and unloaded, silver nanoparticle-containing polymersomes, as indicated.

FIG. 8A is a transmission electron micrograph of a whole E. coli cell after treatment with ampicillin-loaded, silver nanoparticle-containing polymersomes. Scale bar=500 nm. FIG. 8B is a transmission electron micrograph of a whole E. coli cell after treatment with ampicillin-loaded, silver nanoparticle-containing polymersomes. White arrows indicate indentations of the cell membrane where polymersomes are bound, and grey arrow shows polarization of silver nanoparticles within polymersomes. Scale bar=100 nm. FIG. 8C is a transmission electron micrograph of a whole E. coli cell after treatment with ampicillin-loaded, silver nanoparticle-containing polymersomes. White arrow indicates an indentation of the cell membrane where a polymersome is bound. Scale bar=100 nm. FIG. 8D is a transmission electron micrograph of a whole E. coli cell after treatment with ampicillin-loaded, silver nanoparticle-containing polymersomes. Yellow arrows show polarization of silver nanoparticles within polymersomes. Scale bar=100 nm. FIG. 8E is a transmission electron micrograph of a thin bacterial section after treatment with ampicillin-loaded, silver nanoparticle-containing polymersomes. Scale bar=500 nm. FIG. 8F is a transmission electron micrograph of a thin bacterial section after treatment with ampicillin-loaded, silver nanoparticle-containing polymersomes. Arrow indicates region of membrane with little contact with polymersomes. Scale bar=100 nm. FIG. 8G is a transmission electron micrograph of a thin bacterial section after treatment with ampicillin-loaded, silver nanoparticle-containing polymersomes. Scale bar=100 nm. FIG. 8H is a transmission electron micrograph of a thin bacterial section after treatment with ampicillin-loaded, silver nanoparticle-containing polymersomes. Arrow indicates region of membrane with little contact with polymersomes. Scale bar=100 nm.

FIG. 9 is a graph showing the effect of polymersomes on viability CCL-110 human dermal fibroblast cells after 24 hour and 48 hour treatments. 125 μg/mL Ag, 80 μg/mL Amp sample was treated with ampicillin-loaded, silver nanoparticle-containing polymersomes having a silver:ampicillin mass ratio of 1:0.64 at an effective silver concentration of 125 μg/mL; 125 μg/mL Ag, 0 μg/mL Amp sample was treated with ampicillin-free, silver nanoparticle-containing polymersomes at an effective silver concentration of 125 μg/mL; 0 μg/mL Ag, 80 μg/mL Amp sample was treated with free ampicillin at a concentration of 80 μg/mL; and 0 μg/mL Ag, 0 μg/mL Amp sample was treated with suspension buffer. Cell viability was assessed via MTS assays. Values represent the mean±standard deviation, N=3.

FIG. 10 is a schematic of a plain-orifice nozzle (upper diagram) and V-notch shaped orifice nozzle (lower diagram), two single-fluid nozzles that can be used as atomizers in methods of making polymersomes.

### DETAILED DESCRIPTION OF THE INVENTION

The present invention provides polymersomes for the simultaneous delivery of hydrophobic metallic nanoparticles and hydrophilic pharmaceutical agents. The polymersomes contain membranes composed of (1) block copolymers that contain at least one hydrophilic polymer and at least one hydrophobic polymer and (2) hydrophobic metallic nanoparticles. The polymersomes have aqueous lumens that contain an aqueous pharmaceutical agent. The present invention also includes methods of making such polymersomes using atomized delivery of the block copolymers and hydrophobic metallic nanoparticles into an aqueous solution containing the pharmaceutical agent.

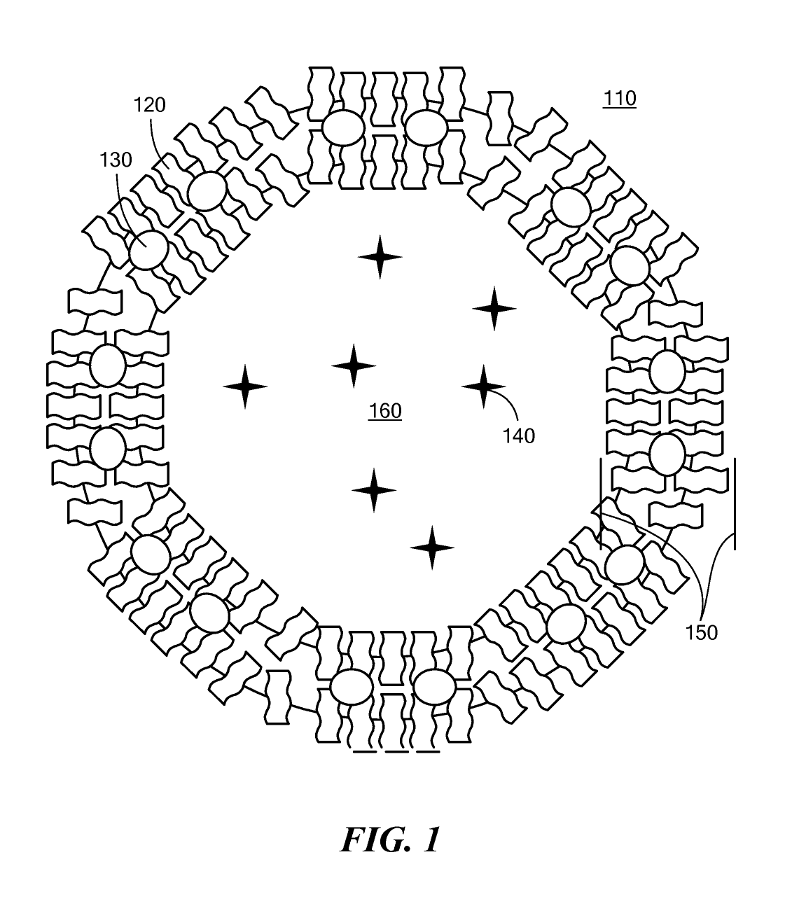

A schematic of a polymersome embodiment of the invention is shown in FIG. 1. The polymersome (110) contains an amphiphilic membrane (150) surrounding an aqueous lumen (160). The membrane contains a block copolymer (120) and one or more hydrophobic metallic nanoparticles (130). In the embodiment shown, the block is a diblock copolymer that includes a hydrophilic polymer and a hydrophobic polymer (not shown). In this embodiment, the diblock copolymers form a bilayer in which the hydrophobic regions make up the interior of the membrane and the hydrophilic regions are exposed to the aqueous milieu in the lumen and external to the polymersome. The hydrophobic nanoparticles are embedded in the hydrophobic interior of the membrane. The aqueous lumen of the polymersome contains the pharmaceutical agent (140).

The invention also encompasses a suspension of such polymersomes in an aqueous solution. A key feature of the suspension is the homogeneity of the polymersome contained therein.

As used herein, “metallic nanoparticle” encompasses nanoparticles containing metals in their pure state, metal oxides, and metal salts.

As used herein, “nanoparticle” refers to a particle having a length in its longest dimension of between about 1 nm and about 999 nm.

The polymersomes may be approximately spherical. Alternatively, the polymersomes may have other shapes, such as rods, flattened sacs, etc. The approximately spherical polymersomes may have a diameter of about 50 nm, about 60 nm, about 70 nm, about 80 nm, about 90 nm, about 100 nm, about 110 nm, about 120 nm, about 150 nm, about 200 nm, from about 50 nm to about 200 nm, from about 100 nm to about 200 nm, from about 150 nm to about 200 nm, from about 60 nm to about 150 nm, from about 70 nm to about 120 nm, from about 80 nm to about 120 nm, from about 90 nm to about 120 nm, from about 95 nm to about 115 nm, or from about 100 nm to about 110 nm. In a suspension of polymersomes, a fraction of the polymersomes may have a diameter as indicated above. For example, at least 50%, at least 60%, at least 70%, at least 80%, at least 90%, at least 95%, at least 96%, at least 97%, at least 98%, or at least 99% of the polymersomes may have diameters as indicated above.

The membranes of the polymersomes may have thickness of about 4 nm, about 5 nm, about 6 nm, about 7 nm, about 8 nm, about 10 nm, about 12 nm, about 15 nm, about 20 nm, about 25 nm, from about 5 nm to about 25 nm, from about 6 nm to about 20 nm, from about 8 nm to about 20 nm, from about 10 nm to about 20 nm, from about 10 nm to about 15 nm, from about 5 nm to about 10 nm, from about 10 nm to about 15 nm, from about 15 nm to about 20 nm, or from about 20 nm to about 25 nm. In a suspension of polymersomes, a fraction of the polymersomes may have a membrane thickness as indicated above. For example, at least 50%, at least 60%, at least 70%, at least 80%, at least 90%, at least 95%, at least 96%, at least 97%, at least 98%, or at least 99% of the polymersomes may have a membrane thickness as indicated above.

The amphiphilic block copolymer may be any copolymer composed of a hydrophilic polymer and a hydrophobic polymer that can spontaneously assemble into a membrane in an aqueous solution. In a preferred embodiment, the amphiphilic block copolymer is a diblock copolymer that has one hydrophilic polymeric region and one hydrophobic polymeric region. In another preferred embodiment, the copolymer is a triblock copolymer that has a first hydrophilic polymeric region, a hydrophobic polymeric region, and a second hydrophilic polymeric region. In polymersomes containing triblock copolymers, one triblock copolymer substitutes for two diblock copolymers and spans the membrane. In a preferred embodiment, the block copolymer is non-toxic. In a preferred embodiment, the block copolymer is biodegradable. In a preferred embodiment, the block copolymer has a hydrophilic fraction (f_{EO}) that supports polymersome formation. For example, the copolymer may have a f_{EO}<50%, <40%, <30%, <20%, <10%, or <5%. The hydrophilic region may contain polyethylene glycol, polyethylene oxide, poly(isocyano-L-alanine-L-lanine, polyacrylic acid, poly(methyloxazoline), poly(4-vinyl pyridine), poly-L-glutamic acid, poly(Nₑ-2-(2-(2-methoxyethoxyl)ethoxy)acetyl-L-lysine, poly(γ-benzyl L-glutamate), or dextran. The polyethylene glycol may be methoxypoly(ethelyne glycol)₅₀₀₀. The hydrophobic region may contain polylactide, poly(lactic acid), poly(ethylethylene), polybutadiene, polycaprolactone, polypropylene sulfide, polystyrene, poly-L-leucine, polyester, poly(butylene oxide), poly(isobutylene), polystyrene-b-poly(isocyanoalanine(2-thiophene-3-ylethyl)amide, poly(2-nitrophenylalanine), poly(γ-methyl-L-caprolactone), or poly(trimethylene carbonate) or hyaluronan. For polymers of chiral molecules, the polymer may contain the D-form, the L-form, or a mixture of the D- and L-forms. For example, the poly(lactic acid) may be poly(D)-(L)-lactic acid_{50,000}. The poly(D)-(L)-lactic acid_{50,000} may have relative percentages of D and L stereoisomers of 10%/90%, 20%/80%, 30%/70%, 40%/60%, 50%/50%, 60%/40%, 70%/30%, 80%/20%, or 90%/10%.

The hydrophobic metallic nanoparticles have a size and shape that allows them to become embedded in the hydrophobic interior of the membrane formed by the amphiphilic block copolymer. The hydrophobic metallic nanoparticles may be approximately spherical, or they may have irregular shapes. The approximately spherical hydrophobic metallic nanoparticles may have diameters of from about 1 nm to about 10 nm, from about 2 nm to about 10 nm, from about 3 nm to about 10 nm, from about 4 nm to about 10 nm, from about 2 nm to about 9 nm, from about 2 nm to about 8 nm, from about 2 nm to about 7 nm, from about 3 nm to about 8 nm, or from about 4 nm to about 7 nm. In preferred embodiments, the hydrophobic metallic nanoparticles have diameters of about 3 nm, about 4 nm, about 5 nm, about 6 nm, or about 7 nm.

The hydrophobic metallic nanoparticles may contain any metal that has a therapeutic benefit. For example, the hydrophobic metallic nanoparticles may contain aluminum, calcium, cerium, copper, gold, iron, lithium, magnesium, manganese, platinum, selenium, silver, titanium, tungsten, vandium, or zinc.

The hydrophobic metallic nanoparticles may have a hydrophobic coating. For example, the hydrophobic metallic nanoparticles may be coated with an alkanethiol, such as decanethiol or dodecanethiol, or another hydrophobic molecule that can be covalently or non-covalently attached to the metallic nanoparticles and is nontoxic. In preferred embodiments, an alkane having a chain length from about 8 to about 16 carbons is covalently attached to the metallic nanoparticles to form the hydrophobic metallic nanoparticles.

The polymersomes may have from about 1 to about 50, from about 1 to about 20, from about 2 to about 20, from about 2 to about 15, from about 3 to about 15, or from about 5 to about 12 hydrophobic metallic nanoparticles per polymersome. In a suspension of polymersomes, a fraction of the polymersomes may have the number of hydrophobic metallic nanoparticles as indicated above. For example, at least 50%, at least 60%, at least 70%, at least 80%, at least 90%, at least 95%, at least 96%, at least 97%, at least 98%, or at least 99% of the polymersomes may have the number of hydrophobic metallic nanoparticles as indicated above.

The polymersomes may contain any pharmaceutical agent that is water-soluble or can be made into a form that is water-soluble. The pharmaceutical agent may be any agent that can be used to treat a disease or medical condition. For example, the pharmaceutical agent may be an agent that can be used to treat bacterial infection, cancer, manic depression, rheumatoid arthritis, and viral infection (AIDS, Herpes). For example, the pharmaceutical agent may be an antitumor agent such as doxorubicin or tamoxifen, or an anti-inflammatory agent. The anti-inflammatory agent may be a corticosteroid, such as beclomethasone, budesonide, dexamethasone, flunisolide, fluticasone propionate, methylprednisolone, prednisolone, prednisone, or triamcinolone. Alternatively, the anti-inflammatory agent may be non-steroidal, such as aceclofenac, acetylsalicylic acid, celecoxib, clonixin, dexibuprofen, dexketoprofen, diclofenac, diflunisal, droxicam, etodolac, etoricoxib, fenoprofen, firocoxib, flufenamic acid, flurbiprofen, ibuprofen, indomethacin, isoxicam, ketoprofen, ketorolac, lornoxicam, loxoprofen, lumiracoxib, meclofenamic acid, mefenamic acid, meloxicam, nabumetone, naproxen, nimesulide, oxaprozin, parecoxib, piroxicam, rofecoxib, salicylic acid, salsalate, sulindac, tenoxicam, tolfenamic acid, valdecoxib, or yolmetin. The pharmaceutical agent may be an antibiotic. For example, the pharmaceutical agent may be amikacin, amoxicillin, amoxicillin/clavulanate, ampicillin, ampicillin/sulbactam, arsphenamine, azithromycin, azlocillin, aztreonam, bacitracin, capreomycin, carbacephem, carbenicillin, cefaclor, cefadroxil, cefalexin, cefalotin, cefamandole, cefazolin, cefdinir, cefditoren, cefepime, cefixime, cefoperazone, cefotaxime, cefoxitin, cefpodoxime, cefprozil, ceftaroline fosamil, ceftazidime, ceftibuten, ceftizoxime, ceftobiprole, ceftriaxone, cefuroxime, chloramphenicol, ciprofloxacin, clarithromycin, clindamycin, clofazimine, cloxacillin, colistin, cycloserine, dalbavancin, dapsone, daptomycin, demeclocycline, dicloxacillin, dirithromycin, doripenem, doxycycline, enoxacin, ertapenem, erythromycin, ethambutol, ethionamide, flucloxacillin, fosfomycin, furazolidone, fusidic acid, gatifloxacin, geldanamycin, gemifloxacin, gentamicin, herbimycin, imipenem/cilastatin, isoniazid, kanamycin, levofloxacin, lincomycin, linezolid, lipopeptide, lomefloxacin, loracarbef, mafenide, meropenem, methicillin, metronidazole, mezlocillin, minocycline, moxifloxacin, mupirocin, nafcillin, nalidixic acid, neomycin, netilmicin, nitrofurantoin, norfloxacin, ofloxacin, oritavancin, oxacillin, oxytetracycline, paromomycin, penicillin, piperacillin, piperacillin/tazobactam, platensimycin, polymyxin B, posizolid, pyrazinamide, quinupristin/dalfopristin, radezolid, rifabutin, rifampicin, rifapentine, rifaximin, roxithromycin, silver sulfadiazine, spectinomycin, spiramycin, streptomycin, sulfacetamide, sulfadiazine, sulfadimethoxine, sulfamethizole, sulfamethoxazole, sulfanilimide, sulfasalazine, sulfisoxazole, teicoplanin, telavancin, telithromycin, ketek, temocillin, tetracycline, thiamphenicol, ticarcillin, ticarcillin/clavulanate, tigecycline, tinidazole, tobramycin, torezolid, trimethoprim, trimethoprim-sulfamethoxazole troleandomycin, or vancomycin.

The polymersome may contain a targeting moiety on its surface. A targeting moiety may be any moiety that enables the polymersome to bind to a cell or tissue that is the intended target for the action of the hydrophilic metallic nanoparticle and pharmaceutical agent. For example, the targeting moiety may be a peptide, polypeptide, protein, or carbohydrate. The targeting moiety may be an antibody, portion of an antibody, or a ligand for a cell-surface receptor. The targeting moiety may be covalently or non-covalently bound to the surface of the polymersome. For example, the targeting moiety can be covalently attached to a hydrophilic portion of a block copolymer of the polymersome.

The invention also encompasses methods of making polymersomes that contain hydrophobic metallic nanoparticles and a pharmaceutical agent. The methods entail providing a suspension of hydrophobic metallic nanoparticles and an amphiphilic block copolymer in an organic solvent, and passing the suspension through an atomizer into an aqueous solution containing a pharmaceutical agent. As the organic suspension passes through the atomizer, hydrophobic interactions cause the block copolymer to form a membrane with the hydrophobic metallic nanoparticles embedded in the interior of the membrane, and the membranes pinch of into polymersomes. Some of the aqueous solution containing the pharmaceutical agent becomes encapsulated in the lumen of the polymersomes, and the organic solvent dissolves into the bulk aqueous solution. The organic solvent and uncaptured pharmaceutical agent are removed from the bulk aqueous solution, for example, by dialysis.

Optimal formation of polymersomes requires the organic solvent to dissolve in the aqueous phase. Preferably, an organic solvent is selected that is miscible in water, as formation of a two-phase solvent system during polymersome formation is to be avoided. For example, the organic solvent may contain tetrahydrofuran, chloroform, methanol, ethanol, isopropanol, or mixtures of these solvents. In addition, to prevent separation of organic and aqueous phases, the volume of the aqueous phase exceeds the volume of the organic phase. For example, the ratio or aqueous: organic phase may be about 5:1, about 10:1, about 20:1, about 50:1, about 100:1, or greater than 100:1.

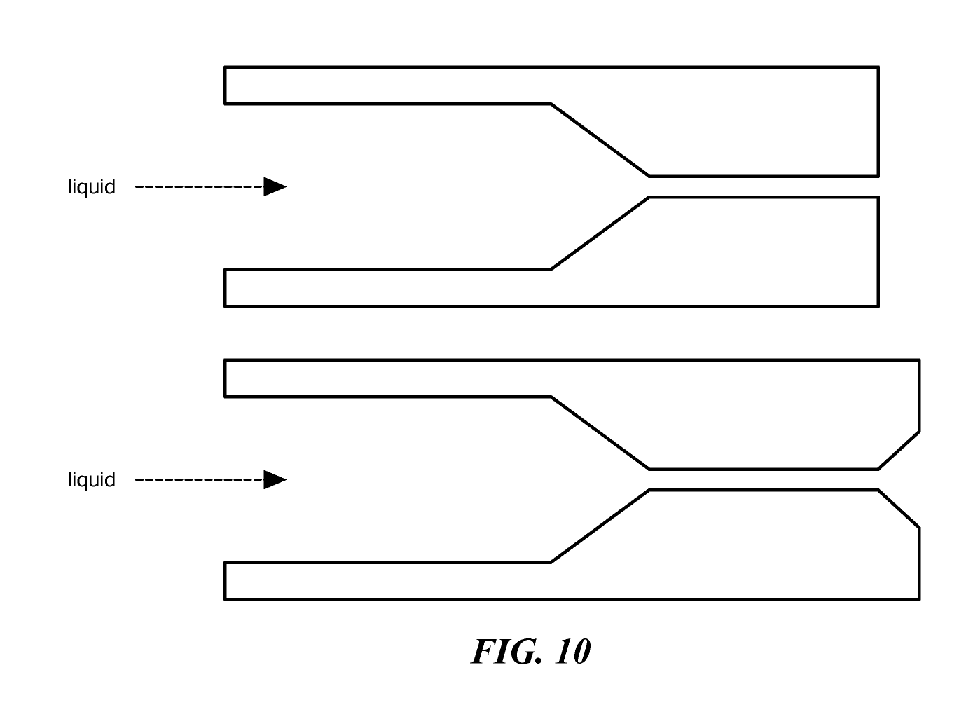

As used herein, an “atomizer” refers to a device that enables emission of a liquid as a plurality of small droplets. The atomizer may be a single-fluid nozzle, two-fluid nozzle, rotary atomizer, ultrasonic atomizer, or electrostatic atomizer. The single-fluid nozzle may be a plain-orifice nozzle, shaped-orifice nozzle, surface-impingement single-fluid nozzle, pressure-swirl single-fluid spray nozzle, solid-cone single-fluid nozzle, or compound nozzle. See, e.g., FIG. 10. The two-fluid nozzle may an internal mix nozzle or an external mix nozzle, The atomizer may have one or more small holes that convert a stream of liquid into small droplets as liquid is forced through the holes. For example, the atomizer may have holes with a diameter of about 500 μm, about 400 μm, about 300 μm, about 200 μm, about 150 μm, about 100 μm, about 50 μm, about 20 μm, about 10 μm, about 5 μm, from about 150 μm to about 200 μm, from about 100 μm to about 150 μm, from about 50 μm to about 100 μm, from about 50 μm to about 100 μm, from about 25 μm to about 50 μm, from about 10 μm to about 25 μm, or from about 5 μm to about 10 μm. For example, the atomizer may be a syringe atomizer. The atomizer can also utilize a single spray nozzle or a compound spray nozzle. The atomizer is preferably made of a material that is stable in the presence of an organic solvent and is capable of withstanding pressurization to drive a stream of the solvent, containing the block copolymer and the hydrophobic nanoparticles, through the atomizer nozzle to form small droplets in the aqueous medium that is to occupy the lumen of the polymersomes.

The rate at which the organic suspension is passed through the atomizer may be varied to optimize polymersome formation. For example, the organic suspension may be passed through the atomizer at a rate of from about 0.05 ml sec⁻¹ to about 0.1 ml sec⁻¹, from about 0.1 ml sec⁻¹ to about 0.2 ml sec⁻¹, from about 0.2 ml sec⁻¹ to about 0.5 ml sec⁻¹, from about 0.5 ml sec⁻¹ to about 1 ml sec⁻¹, from about 1 ml sec⁻¹ to about 5 ml sec⁻¹, or greater than 5 ml sec⁻¹.

The invention also includes methods of treating a disease or condition by administering polymersomes or a suspension containing polymersomes to a subject in need thereof. The polymersomes or suspension of polymersomes may be administered by a parenteral route. The parenteral route may be intravascular administration, peri- and intra-tissue administration, subcutaneous injection or deposition, subcutaneous infusion, intraocular administration, or direct application at or near the site of neovascularization. The polymersomes or suspension of polymersomes may also be delivered by other routes, for example, topically, orally, or intranasally. The disease or condition may be a bacterial infection, cancer, manic depression, rheumatoid arthritis, or viral infection.

The invention also includes the use of polymersomes in diagnostic methods or combined diagnostic-therapeutic methods. In such methods, the polymersome may include an imaging agent capable of detecting and forming an image of population of cells or molecules that serve as markers of a disease or condition. Alternatively, the hydrophobic metallic nanoparticles may be capable of detecting and forming an image of such cells or molecules. The polymersomes may contain both an imaging agent and hydrophobic metallic nanoparticles capable of localizing to, detecting, and forming an image of such cells or molecules.

### EXAMPLES

### Example 1

### Materials and Methods

Particle Synthesis.

The antibiotic solution and silver nanoparticles were encapsulated inside the polymersomes by self-assembly. First, 1 mL of dodecanethiol-functionalized silver nanoparticles (5±2 nm, 0.25% (w/v) in hexane; Sigma-Aldrich, St. Louis, Mo.) was resuspended in 1 mL of tetrahydrofuran (THF; Sigma-Aldrich, St. Louis, Mo.), and subsequently ultrasonicated (Bransonic 2510R-DTH, Emerson Industrial Automation, Danbury, Conn.) to prevent aggregation. 10 mg of the mPEG-PDLLA copolymer (Polyscitech, West Lafayette, Ind.) was added to the mixture, which was again ultrasonicated until the copolymer was completely dissolved. This organic nanoparticle/polymer solution was then injected through a syringe atomizer (MAD300, LMA, San Diego, Calif.) into a 0.01 M solution of PBS (Sigma-Aldrich, St. Louis, Mo.) with or without ampicillin sodium salt (Sigma-Aldrich, St. Louis, Mo.) in a 15 mL glass round bottom tube with a 7×2 mm magnetic stir bar at 500 rpm. Finally, the entire polymersome solution was transferred to a 50 kDa dialysis tube (Spectra/Por Float-A-Lyzer G2, Spectrum Labs, Rancho Dominguez, Calif.) and was allowed to dialyze against pure PBS for 48 hours, with two buffer changes, to remove all traces of the organic solvent and unencapsulated drug.

Particle Characterization.

The size distribution and zeta potential of AgPs were measured using DLS (90Plus Zeta, Brookhaven Instruments, Holtsville, N.Y.) and the software provided by the manufacturer. Ampicillin loading efficiency was determined using a method previously described, 19 based on spectrophotometric optical density measurements at 320 nm (OD320) (SpectraMax M3, Molecular Devices, Sunnyvale, Calif.) of a compound formed by the acidic degradation of ampicillin at 75° C. in pH 5.2 buffer and a trace of copper(II) sulphate pentahydrate (Sigma-Aldrich, St. Louis, Mo.). Ampicillin concentration was measured directly after the synthesis process and after 48 hours of dialysis to remove residual organic solvent. Percentage loading efficiency was calculated as [final concentration]/[initial concentration]×100%. Silver loading was estimated using the following equations. Each AgPs was assumed to contain 9.29±6.07 silver nanoparticle spheres 5 nm in diameter.

(4/3)πr³=65.45 nm³=6.545×10⁻²⁰ cm³ volume per nanoparticle (1)

10.49 g cm⁻³×6.545×10⁻²⁰ cm³=6.866×10⁻¹⁹ g silver per nanoparticle (2)

6.866×10⁻¹⁹ g/107.8682 g mol⁻¹=6.365×10⁻²¹ mol silver per nanoparticle (3)

6.365×10⁻²¹ mol×6.022×10⁻²³ g mol⁻¹=3.833 silver atoms per nanoparticle (4)

Transmission Electron Microscopy

AgPs and cell-particle interactions were visualized using transmission electron microscopy (TEM; JEM-1010, JEOL, Peabody, Mass.). Particles were dried on 300-mesh copper-coated carbon grids (Electron Microscopy Sciences, Hatfield, Pa.) and negatively stained with a 1.5% uranyl acetate solution (Sigma-Aldrich, St. Louis, Mo.). Bacteria were treated with particles for 24 hours, fixed in 4% paraformaldehyde (Sigma-Aldrich, St. Louis, Mo.) for 30 minutes at 4° C., and absorbed on 300-mesh copper-coated carbon grids for imaging. Samples prepared for sectioning were fixed using 3% glutaraldehyde (Sigma-Aldrich, St. Louis, Mo.) and 2% paraformaldehyde, treated with 0.1% tannic acid, and post-fixed with 1% osmium tetroxide (Electron Microscopy Sciences, Hatfield, Pa.) for 30 minutes. Following ethanol gradient dehydration, samples were infiltrated with polymer, cured, sectioned using an ultramicrotome (Reichert-Jung Ultracut E, Reichert Technologies, Buffalo, N.Y.), and absorbed on 200-hex mesh copper-coated carbon grids (Electron Microscopy Sciences, Hatfield, Pa.) for imaging.

Bacteria Transformation.

First, an overnight suspension of E. coli cells (strain K-12 HB101; Bio-Rad, Hercules, Calif.) was pelleted by centrifugation and re-suspended in 250 μL of cold 50 mM calcium chloride (Sigma-Aldrich, St. Louis, Mo.) and placed in an ice bath. After 15 minutes on ice, 10 μg of the plasmid DNA was added, and the cells were returned to the ice bath for an additional 15 minutes. The cells were then heat shocked by placing them in a 42° C. water bath for exactly 45 seconds and then rapidly transferring them back to the ice bath for 2 minutes. This solution was then mixed with 750 μL of Lysogeny broth (LB, Sigma-Aldrich, St. Louis, Mo.). Finally, the complete transformed bacteria solution was streaked for inoculation on a LBagar plate containing 100 μg mL-1 of ampicillin and allowed to incubate overnight at 37° C.

Bacterial Interactions.

For each experimental trial, a single bacterial colony was selected and grown overnight in LB on a shaking incubator set at 200 rpm and 37° C. The overnight bacterial suspension was adjusted by OD₆₀₀ measurements and dilution to possess a final bacterial density of 10⁶ CFU mL⁻¹. 100 μL of the bacterial suspension was then combined with varying AgPs treatment concentrations or controls in a 96 well plate. The final treatment volume in each well was brought up to 100 μL using 0.01 M PBS (e.g. 100 μL treatment+0 μL PBS, 90 μL treatment+10 μL PBS, 80 μL treatment+20 μL PBS, etc.). Control treatments were given 100 μL of PBS to keep the media dilution consistent. The well plate was then allowed to incubate at 37° C. inside a spectrophotometer under static conditions (Spectra-Max Paradigm, Molecular Devices, Sunnyvale, Calif.). OD₆₀₀ measurements were taken every 2 minutes for 24 hours to establish the speed of proliferation and shape of the bacterial growth curve. The differing base OD₆₀₀ values for the various treatment types and concentrations were normalized by subtracting the experimental value from the value of a comparable blank solution.

To assay growth on solid, agar-containing medium, a single ampicillin-resistant colony was selected and grown overnight in Lysogeny broth (LB) on a shaking incubator set at 200 rpm and 37° C. The resulting suspension was then adjusted by OD₆₀₀ measurement and dilution to have a bacterial density of 10⁶ CFU/mL. Following this, solutions containing bacterial densities of 10⁵, 10⁴, and 10³ CFU/mL were also prepared by serial dilution in LB. 100 μL of the bacterial suspensions were then combined with differing concentrations of the AgPs in a 96-well plate, which were then allowed to incubate at 37° C. with 5% carbon dioxide for 24 hours. A 25 μL drop from each well was then plated on a tryptic soy agar plate and allowed to develop overnight in the incubator at 37° C. Finally, the plates were analyzed for the presence or absence of any bacterial growth.

Cytotoxicity.

Cytotoxicity of the polymersome treatments towards human dermal fibroblast cells (Detroit 551 #CCL-110, American Type Culture Collection, Manassas, Va.) was investigated via MTS assays (CellTiter 96® AQueous One Solution Cell Proliferation Assay, Promega, Madison, Wis.). Experiments were carried out in DMEM (American Type Culture Collection, Manassas, Va.) supplemented with 10% fetal bovine serum (American Type Culture Collection, Manassas, Va.) and 1% penicillin-streptomycin (American Type Culture Collection, Manassas, Va.). First, the cells were seeded into a 96 well plate at a density of 5×10³ cells per well (˜1.5×10⁴ cells cm⁻²) with 200 μL of media and allowed to adhere in an incubator at 37° C. and 5% CO₂ for 24 hours. The next day, the media was carefully aspirated from the wells and replaced with a mixture of 100 μL media and 100 μL of AgPs at various dilutions. The final treatment volume in each well was brought up to 100 μL using 0.01 M PBS (e.g. 100 μL treatment+0 μL PBS, 90 μL treatment+10 μL PBS, 80 μL treatment+20 μL PBS, etc.). Control treatments were given 100 μL of PBS to keep the media dilution consistent. Following 24 and 48 hours of incubation at 37° C. and 5% CO₂, the 200 μL treatment/media mixture was removed from each well and replaced with 200 μL of a 1:5 MTS reagent/media mixture. Finally, the plate was returned to the incubator for 4 hours, and the absorbance of each well was subsequently measured using a spectrophotometer (SpectraMax M3, Molecular Devices, Sunnyvale, Calif.) at 490 nm.

Quantification of synergy. The degree of drug synergism was determined using the Bliss Independence Model, in which S=(f_{X0}/f₀₀)(f_{0Y}/f₀₀)−(f_{XY}/f₀₀), where f₀₀ is the wild-type bacteria growth rate in the absence of treatment; f_{X0} and f_{0Y} is the growth rate in the presence of individual drug at X or Y; f_{XY} is the growth rate in the presence of combined drugs X and Y; and S is the degree of synergy.^{22,23} Given that the primary treatment response manifested as a dose-dependent delay in reaching exponential growth phase, here the growth rate was defined as the measured optical density divided by time. The drugs were considered to have a synergistic interaction when S>0, and an antagonistic interaction when S<0.

Statistical Analysis.

All results were presented as the mean±standard deviation unless otherwise noted, and all experiments were repeated at least in triplicate to demonstrate significance (N=3).

### Example 2

### Particle Design, Synthesis, and Characterization

A diblock copolymer of methoxypoly(ethelyne glycol)₅₀₀₀ and poly(D)-(L)-lactic acid_{50,000} (mPEG-PDLLA 5000: 50,000 Da) was utilized for polymersome synthesis. The mPEG block was chosen because it has been documented to confer a “stealth” property to the particles in vivo in order to help prevent premature clearance by the immune system.¹⁷ The racemic mixture of D- to L-lactides in the PDLLA block was optimized to generate polymersomes with a release rate sensitive to changes in temperature.¹⁸ This allows for increased stability (low release) during storage at 4° C., and increased release at physiological temperature.

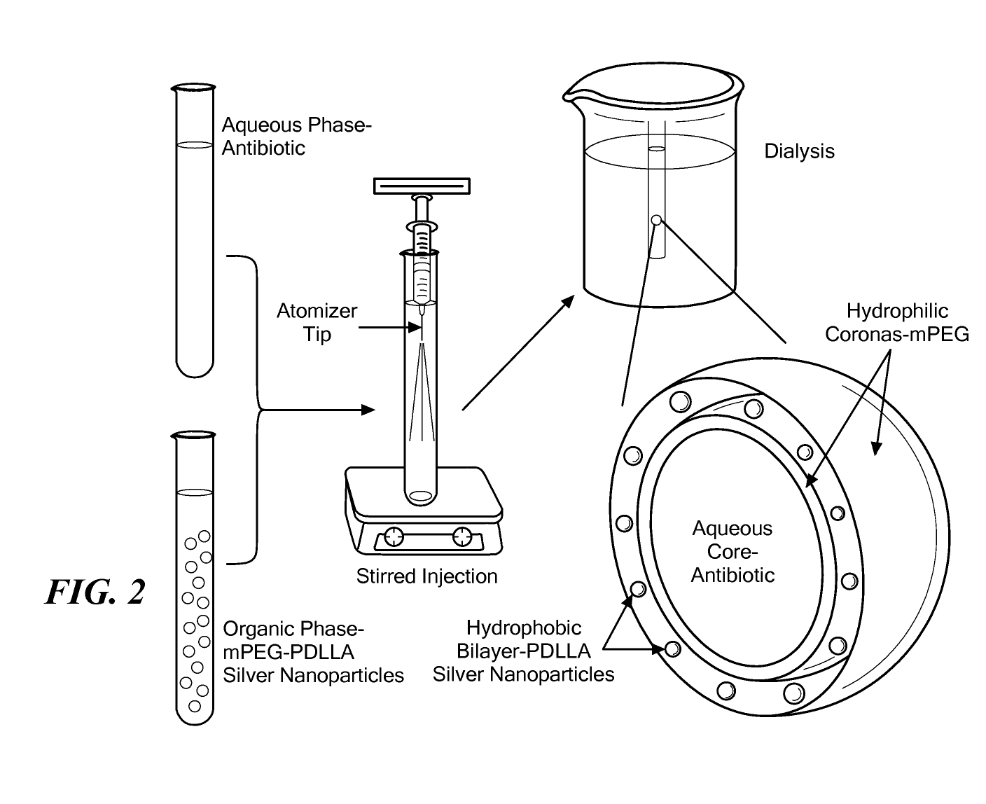

Silver nanoparticle-embedded polymersomes (AgPs) were synthesized using a modified stirred-injection technique. Monodispersed hydrophobic silver nanoparticles 5 nm in diameter were suspended in an organic solvent containing dissolved mPEG-PDLLA. This mixture was injected through a syringe atomizer at high speed into actively stirring phosphate buffered saline (PBS, pH 7.4) containing the antibiotic ampicillin. The resulting suspension was allowed to dialyze against PBS to remove the organic solvent and non-encapsulated drug (FIG. 2).

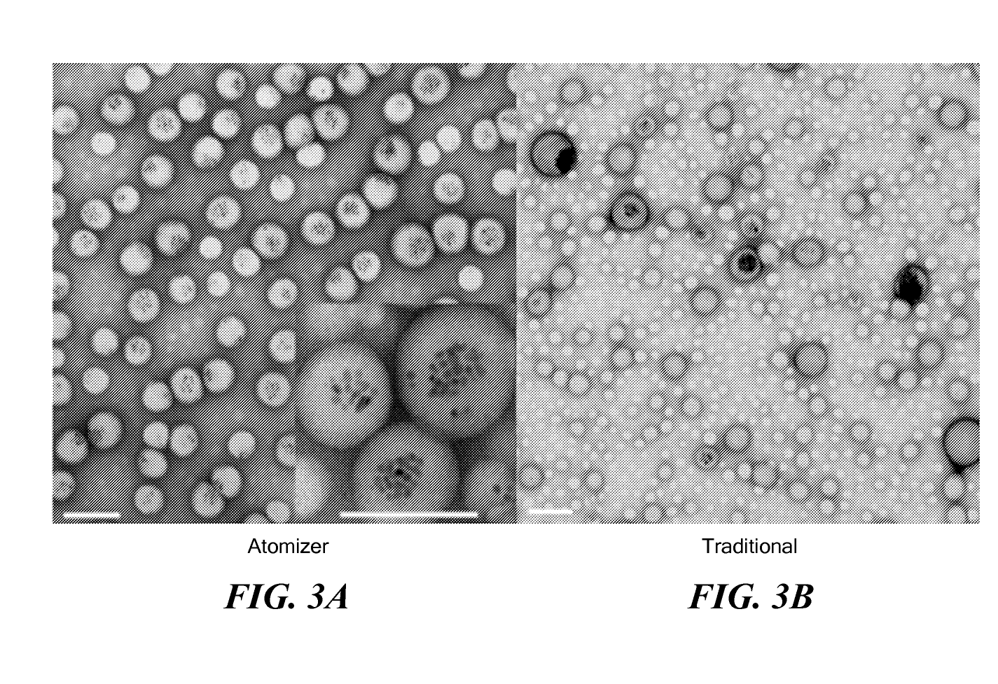

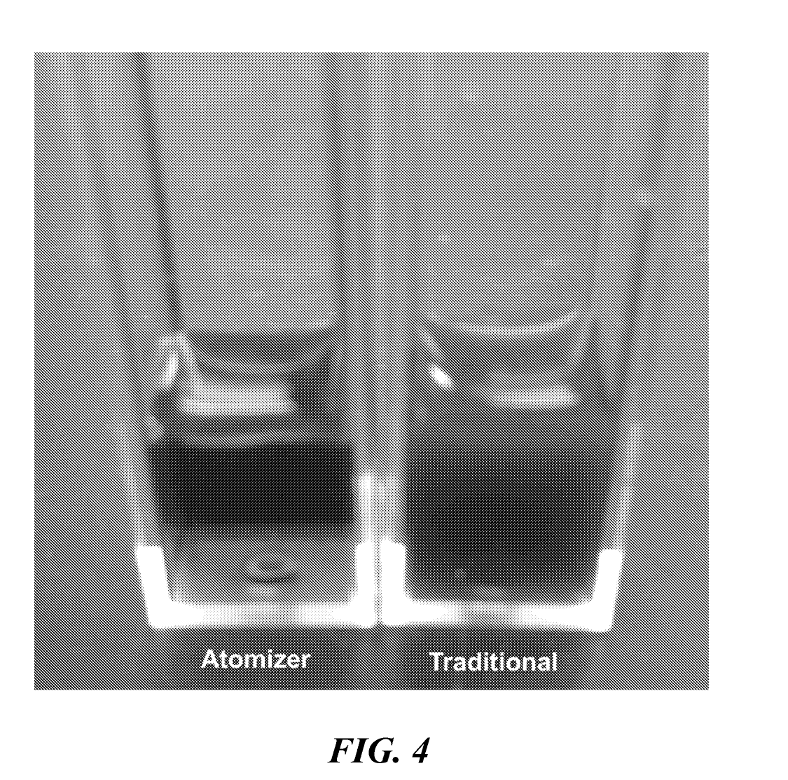

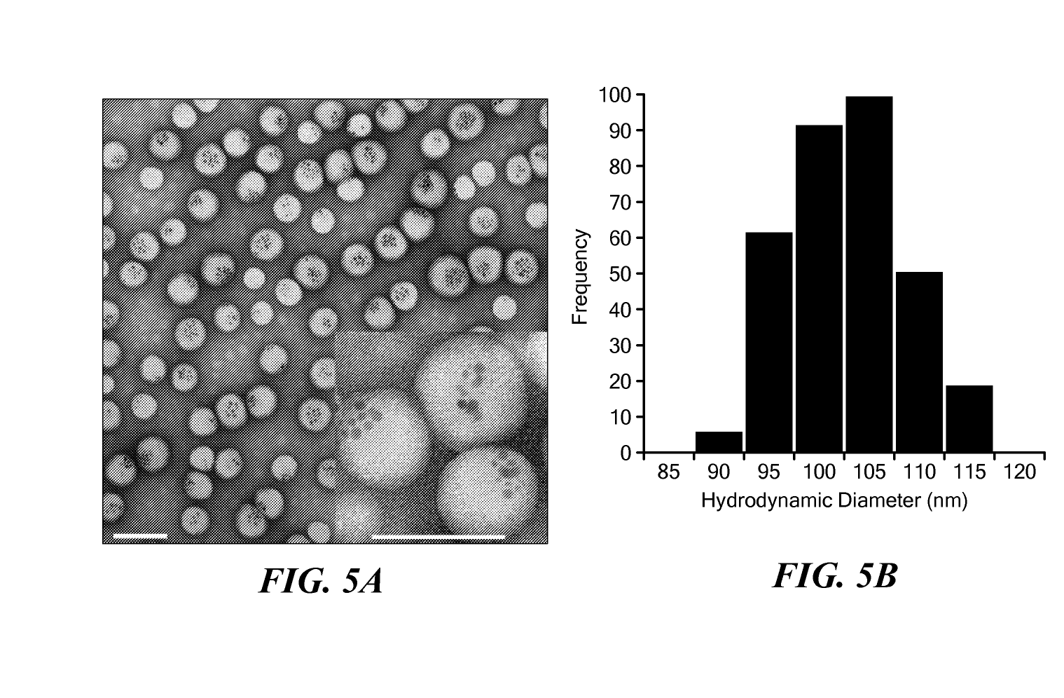

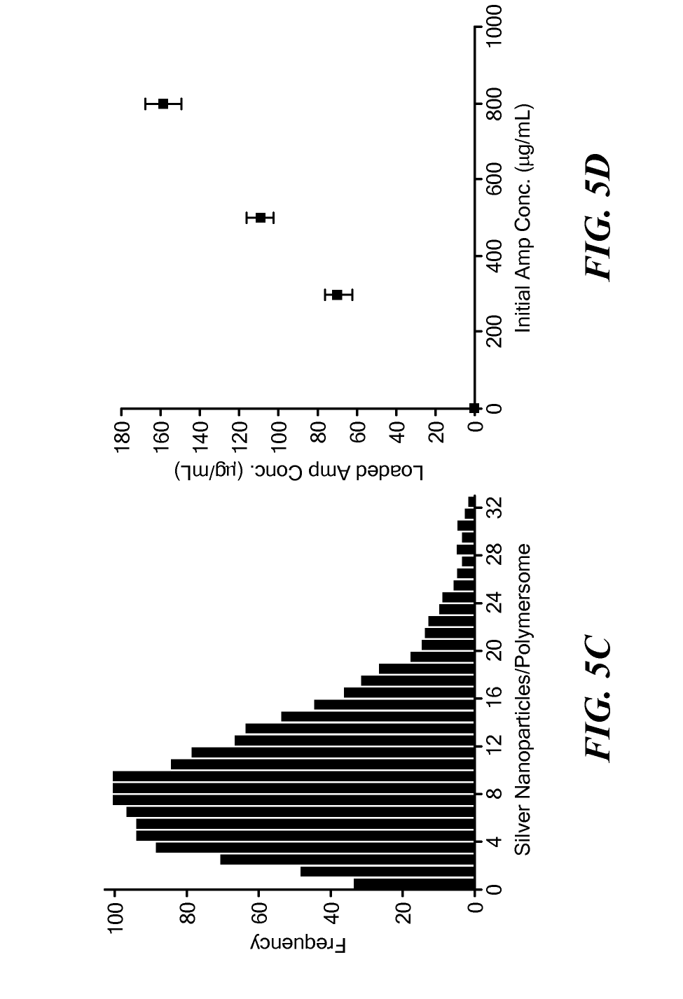

Physicochemical characterization was performed to assess AgPs size, surface charge, and loading. Transmission electron microscopy (TEM) of samples prepared using an atomizer revealed polymersomes of highly uniform size and shape with clusters of silver nanoparticles embedded inside (FIG. 3A). In contrast, polymersomes prepared using a needle were heterogneous in size and in the number of silver nanoparticles per polymersome (FIG. 3B). The homogeneity of the polymersomes prepared using an atomizer is also evident at the macroscopic level by the clarity of the suspension (FIG. 4). The silver clusters frequently appeared as a single layer of nanoparticles that was off-center from the nanoparticle core, suggesting that they may be intercalated into the membrane bilayer (FIG. 5A). Dynamic light scattering (DLS) indicated that the average hydrodynamic diameter was 104.3 nm±15.6 nm (FIG. 5B). The AgPs surface was found to have a near neutral zeta potential of 0.315 mV±1.13 mV at pH 7.4. The number of silver nanoparticles embedded per polymersome was quantified from TEM images. The nanoparticles were shown to load in a normal tailed distribution with an average of 9.29±6.07 silver nanoparticles per polymersome (FIG. 5C). The mass of silver loaded was estimated using the density of silver and the volume of a 5 nm sphere (Table 1).

Three different AgPs formulations were synthesized to contain different concentrations of ampicillin. The loading efficiency of ampicillin in the aqueous phase was measured by spectrophotometry as previously described.¹⁹ The final ampicillin concentration following particle dialysis was determined to be 70 μg mL−1±7.0 μg mL−1, 110 μg mL−1±7.2 μg mL−1, and 160 μg mL−1±9.0 μg mL−1, corresponding to a loading efficiency of 23%, 22%, and 20%, respectively (FIG. 5D). The final silver-to-ampicillin molecular ratio for the different AgPs formulations was 1:0.28, 1:0.44, and 1:0.64, respectively.

### Example 3

### Bacterial Growth Inhibition

E. coli is a Gram-negative, rod-shaped bacterium which has been extensively investigated in the laboratory for over 60 years, making it one of the most widely studied prokaryotic organisms and thus ideal for a proof-of-concept application. First, E. coli cells were transformed with a plasmid containing the bla gene encoding for the enzyme TEM-1 β-lactamase using calcium chloride and heat-shock.²⁰ TEM-1 is the most common β-lactamase found in enterobacteriaceae, and confers resistance to multiple antibiotics including the narrow-spectrum cephalosporins, cefamandole, cefoperazone, and all of the penicillins except for temocillin.²¹

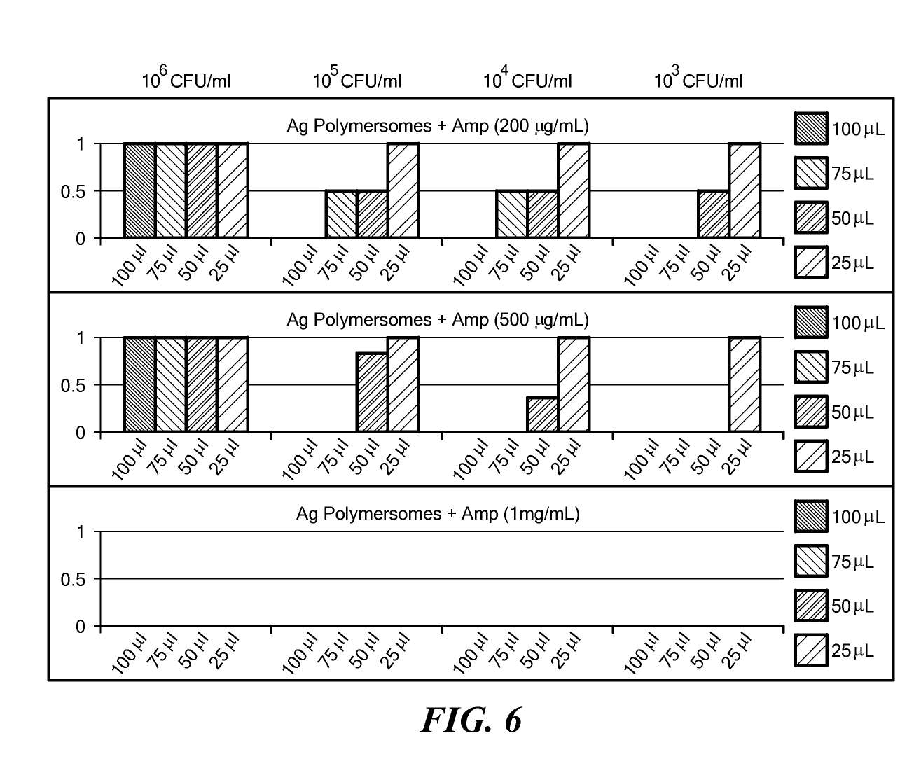

The effectiveness of AgPs at preventing bacterial growth was analyzed in a plating assay. AgPs were made in the presence of different concentrations of ampicillin. Varying concentrations of such AgPs were incubated for 24 hours with cultures of bacterial cells at different culture densities, and samples from the cultures were tested for growth on agar medium. Moderate bactericidal action was observed at higher concentrations of AgPs and lower bacterial seeding densities (FIG. 6).

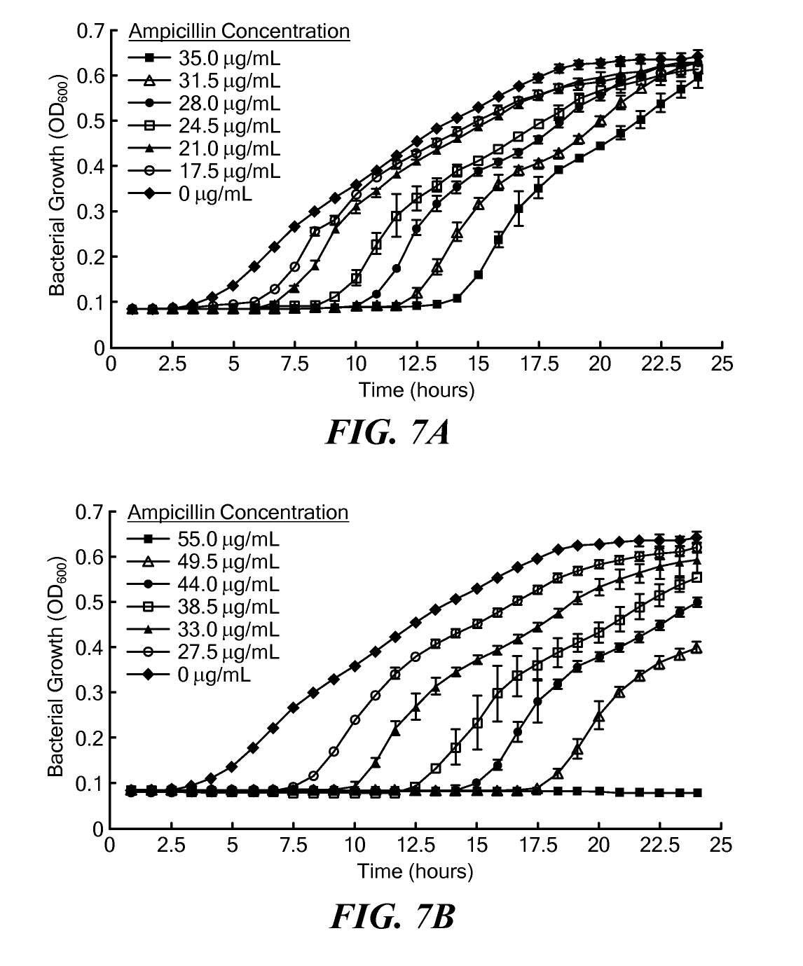

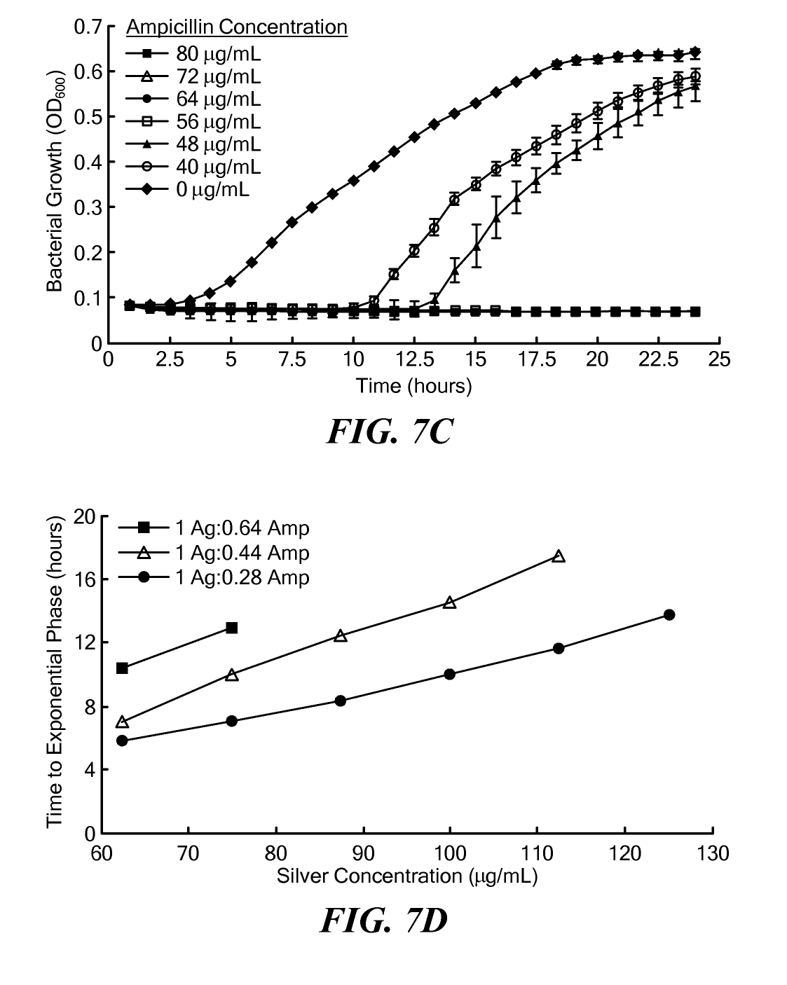

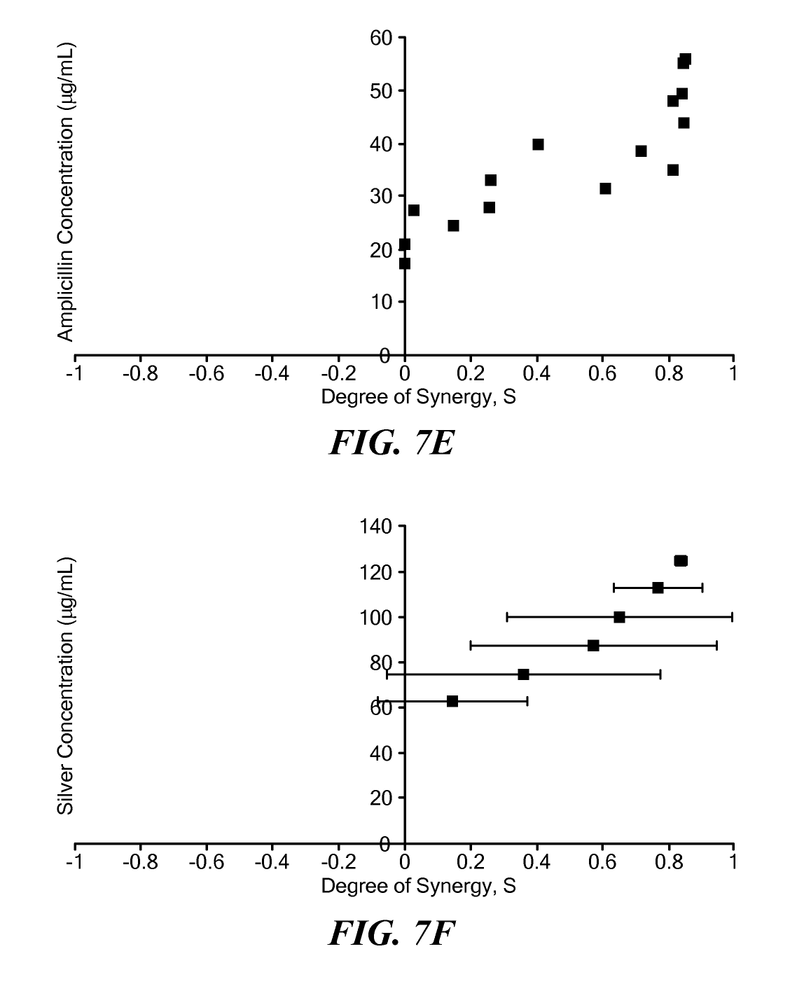

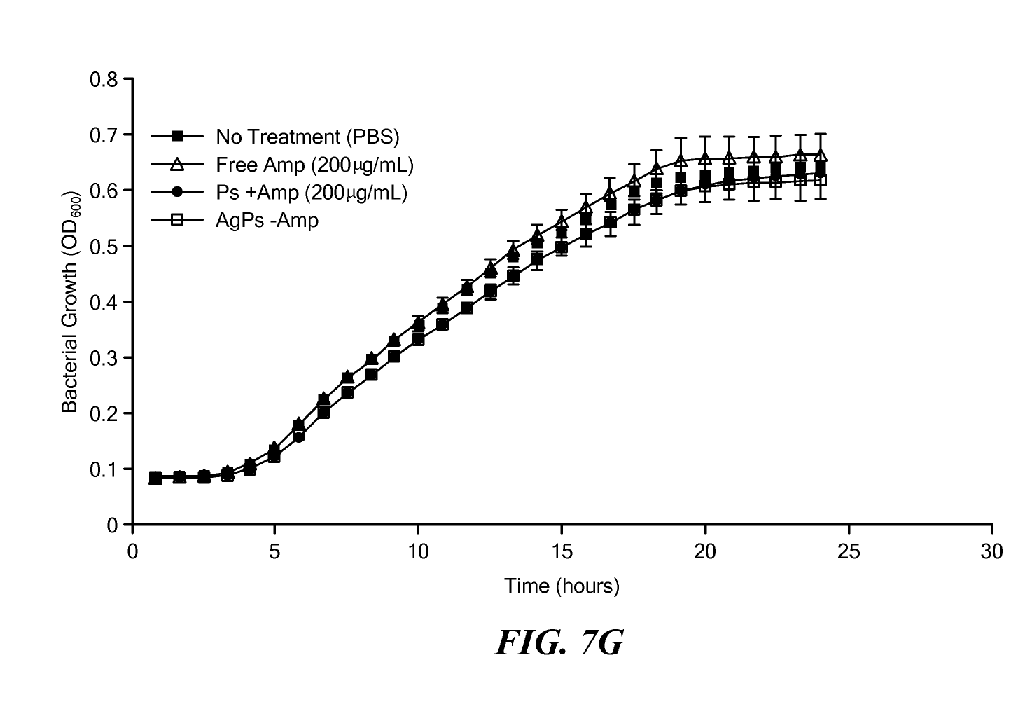

The growth and proliferation of a 10⁶ colony forming units mL⁻¹ (CFU mL⁻¹) suspension of ampicillin-resistant E. coli was examined by measuring the optical density at 600 nm (OD₆₀₀) for 24 hours following treatment with volumes of AgPs containing a silver:ampicillin (Ag:Amp) ratio of 1:0.28 (FIG. 7A), 1:0.44 (FIG. 7B), or 1:0.64 (FIG. 7C). Ampicillin-loaded AgPs displayed significant bacteriostatic action against the E. coli, manifesting as a delay in the time taken to reach exponential growth phase. This response was dose-dependent, with higher concentrations of ampicillin producing a longer delay in bacterial growth. Bacteria treated with ampicillin concentrations above 55 μg mL⁻¹ failed to proliferate within 48 hours. In the absence of silver nanoparticles, no bacteriostatic effect was observed for all ampicillin concentrations tested. This suggests that the presence of silver potentiates the therapeutic efficacy of ampicillin. AgPs without ampicillin likewise produced no therapeutic benefit. Additionally, no significant differences were observed between bacteria treated with free ampicillin (200 μg mL⁻¹), PBS, AgPs without ampicillin, and ampicillin loaded polymersomes (200 μg mL⁻¹) without silver nanoparticles (FIG. 7G). When bacteria were treated with suboptimal concentrations of AgPs, bacterial growth was always observed within 17 hours. The time to exponential phase was found to vary with both silver concentration and ampicillin loading (FIG. 7D).

TABLE 1

| | Per ml AgPs | Per silver | |
|---|---|---|---|
| Silver | nanoparticle | nanoparticle | Per average AgPs |
| g | 2.5 × 10⁻⁴ | 6.9 × 10⁻¹⁹ | 6.4 × 10⁻¹⁸ ± 4.1 × 10⁻¹⁸ |
| mol | 2.3 × 10⁻⁶ | 6.4 × 10⁻²¹ | 5.9 × 10⁻²⁰ ± 3.8 × 10⁻²⁰ |
| atoms | 1.4 × 10¹⁸ | 3.8 × 10³ | 3.6 × 10⁴ ± 2.3 × 10⁴ |

A Bliss Model was utilized to determine the degree of synergy for different silver and ampicillin combinations.^{22,23} Drug interactions were found be synergistic (S>0) in all cases where ampicillin was supplied at concentrations of 24 μg mL⁻¹ and above (FIG. 7E). At lower concentrations, no synergism was observed (S=0). The degree of synergy was dose-dependent and increased with both silver and ampicillin concentrations. The therapeutic benefit of ampicillin reached a plateau at 50 μg mL⁻¹ over a range of silver concentrations due to complete inhibition of bacterial growth. When the silver concentration was held constant, the degree of synergism was directly determined by the amount of ampicillin loaded. This phenomenon is highlighted in FIG. 7F, where the bars indicate the range of synergism that can be achieved by varying ampicillin loading at a fixed silver concentration.

### Example 4

### Cell-Particle Interactions

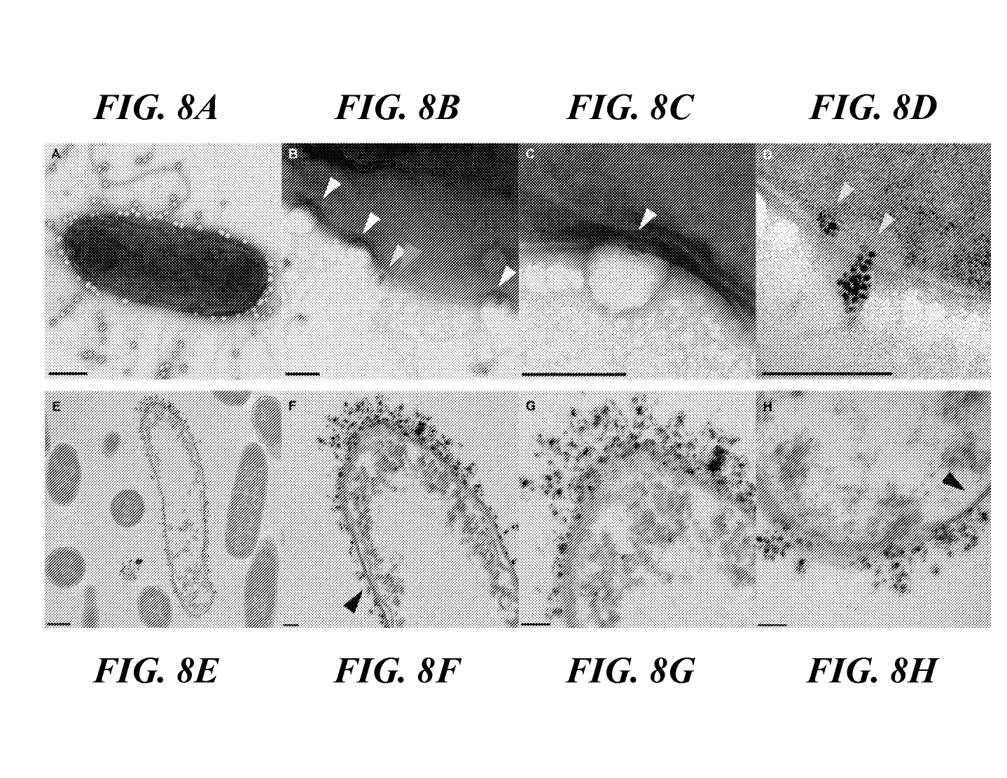

Interactions between E. coli and AgPs were visualized using TEM (FIG. 8A-H). Indentation of the bacterial cell membrane was observed in regions of AgPs contact (FIG. 8B, C; white arrows). Silver nanoparticles inside AgPs appeared to be polarized in an orientation perpendicular to the bacterial cell membrane, suggestive of hydrophobic interactions with the outer cell membrane (FIG. 8B, D). In order to assess physical intracellular changes caused by AgPs, cells treated with an intermediate particle concentration (44 μg mL⁻¹ Amp, 1 Ag:0.44 Amp) for 24 hours were sectioned. Bacteria in contact with AgPs displayed significant protein aggregation and diffuse widening of the cell envelope (FIGS. 8E-H). This phenomenon has been observed by others following silver ion treatment, and has been shown to correlate with increased membrane permeability and protein misfolding due to disulfide bond disruption.^{22,24,25} Regions of the cell envelope with little to no AgPs contact appeared morphologically normal (FIGS. 8F-H; black arrows).

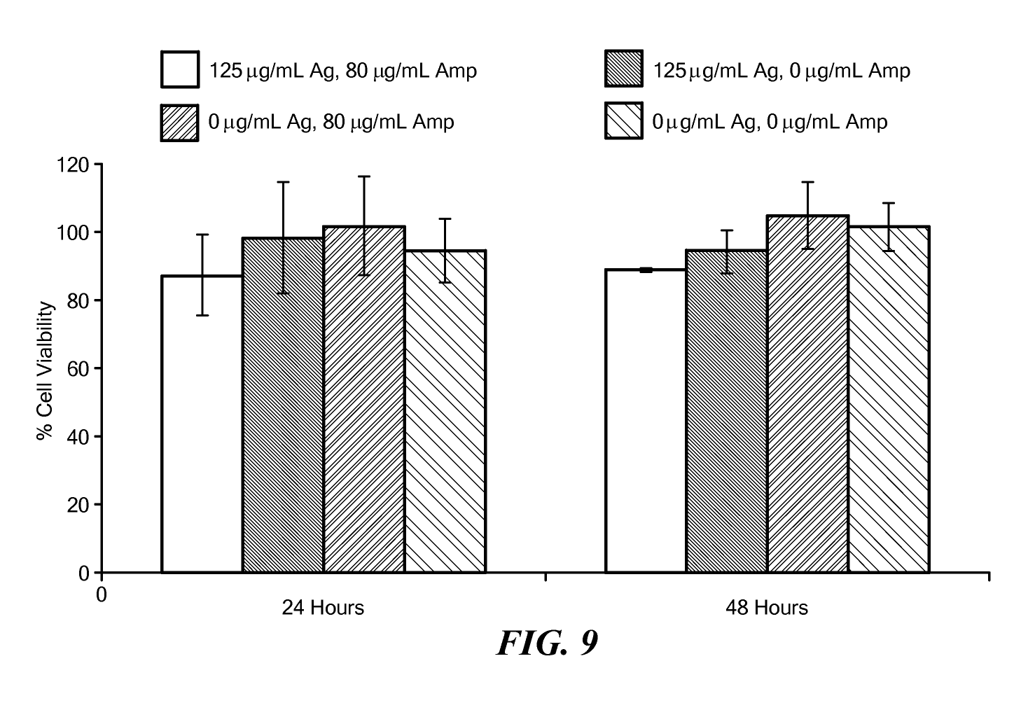

The cytotoxicity of AgPs to mammalian cells was investigated using CCL-110 human dermal fibroblasts (FIG. 9). Cells were treated with different concentrations of AgPs for 24 or 48 hours, and cell viability was measured using MTS assays. No significant cytotoxicity was observed over a range of 0-80 μg mL⁻¹ ampicillin and 0-125 μg mL⁻¹ silver.

### REFERENCES

1 G. Taubes, The bacteria fight back, Science, 2008, 321, 356.
2 L. B. Rice, Emerging issues in the management of infections caused by multidrug-resistant gram-negative bacteria, Cleve Clin. J. Med., 2007, 74, S12-S20.
3 Antibiotic Resistance Threats in the United States, Centers for Disease Control and Prevention, 2013.
4 J. M. Pages, C. E. James and M. Winterhalter, The porin and the permeating antibiotic: a selective diffusion barrier in Gram-negative bacteria, Nat. Rev. Microbiol., 2008, 6, 893.
5 S. R. Norrby, C. E. Nord and R. Finch, Lack of development of new antimicrobial drugs: a potential serious threat to public health, The Lancet Infectious Diseases, 2005, 5(2), 115-119.
6 J. S. Lee, Polymersomes for drug delivery: Design, formation and characterizations, J. Controlled Release, 2012, 161, 473-483.
7 N. Hoiby, T. Bjarnsholt, M. Givskov, S. Molin and O. Ciofu, Antibiotic resistance of bacterial biofilms, Int. J. Antimicrob. Agents, 2010, 35, 22-332.
8 M. C. Nacucchio, M. J. Bellora, D. O. Sordelli and M. D'Aquino, Enhanced liposome-mediated activity of piperacillin against staphylococci, Antimicrob. Agents Chemother., 1985, 27, 137-139.
9 M. Alhajlan, M. Alhariri and A. Omri, Efficacy and safety of liposomal clarithromycin and its effect of Pseudomonas aeruginosa virulence factors, Antimicrob. Agents Chemother., 2013, 57, 2694-2704.
10 B. M. Geilich and T. J. Webster, Reduced adhesion of Staphylococcus aureus to ZnO/PVC nanocomposites, Int. J. Nanomed., 2013, 8, 1177-1184.
11 L. N. Magner, in Hippocrates and the Hippocratic Tradition. A History of Medicine, ed. J. Duffy, Marcel Dekker, Inc., NYC, 1992, p. 393.
12 S. Shrivastava, T. Bera, A. Roy, G. Singh, P. Ramachandrarao and D. Dash, Characterization of enhanced antibacterial effects of novel silver nanoparticles, Nano, 2007, 18, 225103.
13 A. R. Shahverdi, A. Fakhimi, H. R. Shahverdi and S. Minaian, Synthesis and effect of silver nanoparticles on the antibacterial activity of different antibiotics against Staphylococcus aureus and Escherichia coli, J. Nano., 2007,3(2), 168-171.
14 P. Li, J. Li, C. Wu, Q. Wu and J. Li, Synergistic antibacterial effects of β-lactam antibiotic combined with silver nanoparticles, Nano., 2005, 16(9), 1912.
15 A. M. Fayaz, K. Balaji, M. Girilal, R. Yadav, P. T. Kalaichelvan and R. Venketesan, Biogenic Synthesis of Silver Nanoparticles and their Synergistic Effect with Antibiotics: A Study Against Gram-Positive and Gram-Negative Bacteria, Nanomedicine: Nanotechnology, Biology and Medicine, 2010, 6(1), 103-109.
16 Y. Li, X. Chen and N. Gu, Computational Investigation of Interaction between Nanoparticles and Membranes: Hydrophobic/Hydrophilic Effect, J. Phys. Chem. B, 2008, 112, 16647-16653.
17 M. D. Scott, K. L. Murad, F. Koumpouras, M. Talbot and J. W. Eaton, Chemical camouflage of antigenic determinants: Stealth erythrocytes, Proc. Natl. Acad. Sci. U.S.A, 1997, 94, 566-7571.
18 M. Santin, Strategies in Regenerative Medicine: Integrating Biology with Materials Design, Springer, 2009, p. 62.
19 J. W. G. Smith, G. E. Grey and V. J. Patel, Spectrophotometric Determination of Ampicillin, Analyst, 1967, 92, 247-252.
20 A. Froger and J. E. Hall, Transformation of Plasmid DNA into E. coli Using the Heat Shock Method, J Vis Exp., 2007, 6, 253.
21 A. Matagne, J. Lamotte-Brasseur and J. M. Frere, Catalytic properties of class A beta-lactamases: efficiency and diversity, Biochem. J., 1998, 330(2), 581-598.
22 J. R. Morones-Ramirez, J. A. Winkler, C. S. Spina and J. J. Collins, Silver Enhances Antibiotic Activity Against Gram-Negative Bacteria, Sci. Transl. Med., 2013, 5(190), 190ra81.
23 M. Hegreness, N. Shoresh, D. Damian, D. Hartl and R. Kishony, Accelerated evolution of resistance in multidrug environments, Proc. Natl. Acad. Sci. U.S.A, 2008, 105, 13977.
24 Q. L. Feng, J. Wu, G. Q. Chen, F. Z. Cui, T. N. Kim and J. O. Kim, A mechanistic study of the antibacterial effect of silver ions on Escherichia coli and Staphylococcus aureus, J. Biomed. Mater. Res., 2000, 52(4), 662-668.
25 S. Y. Liau, D. C. Read, W. J. Pugh, J. R. Furr and A. D. Russell, Interaction of silver nitrate with readily identifiable groups: relationship to the antibacterial action of silver ions, Lett. Appl. Microbiol., 1997, 25(4), 279-283.
26 H. Nikaido and M. Vaara, Molecular Basis of Bacterial Outer Membrane Permeability, Microbiological Reviews, 1985, 49(1), 1-32.
27 M. T. Silva and J. C. Sousa, Ultrastructure of the Cell Wall and Cytoplasmic Membrane of Gram-Negative Bacteria with Different Fixation Techniques, J. Bacteriol., 1973, 113(2), 953-962.
28 C. R. Raetz, Biochemistry of Endotoxins, Annu. Rev. Biochem., 1990, 59(1), 129-170.
29 S. P. Lim and H. Nikaido, Kinetic Parameters of Efflux of Penicillins by the Multidrug Efflux Transporter AcrAB-Tolc of Escherichia coli, Antimicrob. Agents Chemother., 2010, 54(5), 1800-1806.
30 C. Chervaux, N. Sauvonnet, A. Le Clainche, B. Kenny, A. L. Hung, J. K. Broome-Smith and I. B. Holland, Secretion of active beta-lactamase to the medium mediated by the Escherichia coli haemolysin transport pathway, Mol. Gen. Genet., 1995, 249(2), 237-245.
31 J. S. Kim, E. Kuk, K. N. Yu, J. Kim, S. J. Park, H. J. Lee, S. H. Kim, Y. K. Park, Y. H. Park, C. Hwang, et al. Antimicrobial Effects of Silver Nanoparticles, Nanomedicine: Nanotechnology, Biology and Medicine, 2007, 3(1), 95-101.
32 I. Sondi and B. Salopek-Sondi, Silver Nanoparticles as Antimicrobial Agent: A Case Study on E. coli as a model for Gram-negative bacteria, J. Colloid Interface Sci., 2004, 275(1), 177-182.
33 K. Kalishwaralal, S. BarathManiKanth, S. R. K. Pandian, V. Deepak and S. Gurunathan, Silver Nanoparticles Impede the Biofilm Formation by Pseudomonas aeruginosa and Staphylococcus epidermidis, Colloids Surf., B, 2010, 79(2), 340-344.
34 D. Roe, B. Karandikar, N. Bonn-Savage, B. Gibbins and J. Roullet, Antimicrobial Surface Functionalization of Plastic Catheters by Silver Nanoparticles, J. Antimicrob. Chemother., 2008, 61(4), 869-876.
35 T. Mah, Biofilm-Specific Antibiotic Resistance, Future Microbiology, 2012, 7(9), 1061.
36 P. V. AshaRani, G. L. K. Mun, M. P. Hande and S. Valiyaveettil, Cytotoxicity and Genotoxicity of Silver Nanoparticles in Human Cells, ACS Nano, 2009, 3(2), 279-290.
37 M. Ahamed, M. S. AlSalhi and M. K. J. Siddiqui, Silver nanoparticle applications and human health, Clin. Chim. Acta, 2010, 411(23-24), 1841-1848.
38 R. Lima, A. B. Seabra and N. Duran, Silver nanoparticles: a brief review of cytotoxicity and genotoxicity of chemically and biogenically synthesized nanoparticles, J. Appl. Toxicol., 2012, 32, 867-879.

What is claimed is:

1. A polymersome comprising:
(a) a membrane having a hydrophobic interior and hydrophilic inner and outer surfaces, the membrane comprising:
(i) an amphiphilic block copolymer comprising a hydrophobic block and a hydrophilic block, wherein the interior of the membrane comprises the hydrophobic block and the inner and outer surfaces of the membrane comprise the hydrophilic block; and
(ii) one or more hydrophobic metallic nanoparticles disposed in the interior of the membrane; and
(b) an aqueous lumen comprising a pharmaceutical agent.

2. The polymersome of claim 1, wherein the hydrophobic metallic nanoparticles comprise one or more metals selected from the group consisting of aluminum, calcium, cerium, copper, gold, iron, lithium, magnesium, manganese, platinum, selenium, silver, titanium, tungsten, vanadium, and zinc.

3. The polymersome of claim 1, wherein the hydrophobic metallic nanoparticles have an average diameter of from about 2 nm to about 10 nm.

4. The polymersome of claim 1, wherein the hydrophobic metallic nanoparticles are functionalized with an alkanethiol.

5. The polymersome of claim 1, wherein the amphiphilic block copolymer is a diblock copolymer.

6. The polymersome of claim 5, wherein the diblock copolymer comprises polyethylene glycol or a derivative thereof.

7. The polymersome of claim 5, wherein the diblock copolymer comprises poly(lactic acid).

8. The polymersome of claim 1, wherein the pharmaceutical agent is an antibiotic.

9. The polymersome of claim 1, wherein the polymersome has a diameter from about 80 nm to about 120 nm.

10. The polymersome of claim 1, wherein the hydrophobic metallic nanoparticles comprise silver.

11. The polymersome of claim 1, wherein the polymersome comprises from about 1 to about 20 nanoparticles.

12. The polymersome of claim 1, wherein the mass ratio of nanoparticles to pharmaceutical agent in the polymersome is from about 1:1 to about 5:1.

13. An aqueous suspension comprising a plurality of the polymersomes of claim 1, wherein at least 90% of the polymersomes have a diameter in the range from about 80 nm to about 120 nm.

14. The aqueous suspension of claim 13 that is formulated for injection into a subject.

15. A method of making polymersomes, the method comprising the steps of:
(a) providing a suspension of hydrophobic metallic nanoparticles and an amphiphilic block copolymer in an organic solvent; and
(b) passing the suspension through an atomizer into an aqueous solution comprising a pharmaceutical agent.

16. The method of claim 15, wherein the hydrophobic metallic nanoparticles comprise one or more metals selected from the group consisting of aluminum, calcium, cerium, copper, gold, iron, lithium, magnesium, manganese, platinum, selenium, silver, titanium, tungsten, vanadium, and zinc.

17. The method of claim 15, wherein the hydrophobic metallic nanoparticles have an average diameter of from about 2 nm to about 10 nm.

18. The method of claim 15, wherein the hydrophobic metallic nanoparticles are functionalized with an alkanethiol.

19. The method of claim 15, wherein the amphiphilic block copolymer is a diblock copolymer.

20. The method of claim 19, wherein the diblock copolymer comprises polyethylene glycol or a derivative thereof.

21. The method of claim 19, wherein the diblock copolymer comprises poly(lactic acid).

22. The method of claim 15, wherein the pharmaceutical agent is an antibiotic.

23. The method of claim 15, wherein at least 90% of the polymersomes made by the method have a diameter in the range from about 80 nm to about 120 nm.

24. The method of claim 15, wherein the hydrophobic metallic nanoparticles comprise silver.

25. The method of claim 15, wherein at least 90% of the polymersomes made by the method comprise from about 1 to about 20 nanoparticles per polymersome.

26. The method of claim 15, wherein the mass ratio of metallic nanoparticles to pharmaceutical agent in the nanoparticles is from about 1:1 to about 5:1.

27. A method of treating a disease or condition, the method comprising administering to a subject in thereof an aqueous suspension of polymersomes, the polymersomes comprising:
(a) a membrane having a hydrophobic interior and hydrophilic inner and outer surfaces, the membrane comprising:
(i) an amphiphilic block copolymer comprising a hydrophobic block and a hydrophilic block, wherein the interior of the membrane comprises the hydrophobic block and the inner and outer surfaces of the membrane comprise the hydrophilic block; and
(ii) one or more hydrophobic metallic nanoparticles in the interior of the membrane; and
(b) an aqueous lumen comprising a pharmaceutical agent.

28. The method of claim 27, wherein the aqueous suspension of polymersomes is administered by a parenteral route.

29. The method of claim 28, wherein the parenteral administration route is selected from the group consisting of intravascular administration, intravenous administration, peri- and intra-tissue administration, subcutaneous injection or deposition, subcutaneous infusion, intraocular administration, and direct application at or near a site of neovascularization.

30. The method of claim 27, wherein the disease or condition is selected from the group consisting of bacterial infection, viral infection, cancer, inflammation, and mental disorders.

31. The method of claim 27, wherein the polymerosomes further comprise a targeting moiety covalently attached to the outer surface of the membrane.

32. A kit for treating or preventing a microbial infection, the kit comprising:
(a) an aqueous suspension of polymersomes, the polymersomes comprising:
(i) a membrane having a hydrophobic interior and hydrophilic inner and outer surfaces, the membrane comprising:
(A) an amphiphilic block copolymer comprising a hydrophobic block and a hydrophilic block, wherein the interior of the membrane comprises the hydrophobic block and the inner and outer surfaces of the membrane comprise the hydrophilic block; and
(B) one or more hydrophobic metallic nanoparticles in the interior of the membrane; and
(ii) an aqueous lumen comprising a pharmaceutical agent; and
(b) instructions for use.

33. A kit for preparing an aqueous suspension of polymersomes, the polymersomes comprising:
(i) a membrane having a hydrophobic interior and hydrophilic inner and outer surfaces, the membrane comprising:
(A) an amphiphilic block copolymer comprising a hydrophobic block and a hydrophilic block, wherein the interior of the membrane comprises the hydrophobic block and the inner and outer surfaces of the membrane comprise the hydrophilic block; and
(B) one or more hydrophobic metallic nanoparticles in the interior of the membrane; and
(ii) an aqueous lumen comprising a pharmaceutical agent;
the kit comprising:
(a) a solution of said amphiphilic block copolymer in an organic solvent;
(b) an atomization device; and
(c) instructions for performing the method of claim 15.

34. The kit of claim 33 further comprising:
(d) a plurality of hydrophobic metallic nanoparticles; and optionally
(e) a pharmaceutical agent and/or an imaging agent.

35. A method of imaging a population of cells or molecules in a subject, the method comprising administering to a subject in thereof an aqueous suspension of polymersomes, the polymersomes comprising:
(a) a membrane having a hydrophobic interior and hydrophilic inner and outer surfaces, the membrane comprising:
(i) an amphiphilic block copolymer comprising a hydrophobic block and a hydrophilic block, wherein the interior of the membrane comprises the hydrophobic block and the inner and outer surfaces of the membrane comprise the hydrophilic block; and
(ii) one or more hydrophobic metallic nanoparticles in the interior of the membrane; and
(b) an aqueous lumen comprising a pharmaceutical agent and/or an imaging agent; wherein the hydrophobic metallic nanoparticles and/or the imaging agent is capable of detection and forming an image of said population of cells or molecules in said subject.

36. The method of claim 35, wherein the polymersomes further comprise a targeting moiety covalently attached to the outer surface of the membrane.
